(19) United States
(12) Patent Application Publication
(10) Pub. No.: US 2024/0189509 A1
(43) Pub. Date: Jun. 13, 2024

(54) SYNCOPE PREDICTION USING CARDIAC ACCELERATION INFORMATION

(71) Applicant: Cardiac Pacemakers, Inc., St. Paul, MN (US)

(72) Inventors: Mojgan Goftari, Shoreview, MN (US); Jonathan Bennett Shute, Eagan, MN (US); Pramodsingh Hirasingh Thakur, Woodbury, MN (US); Ashley Moriah Jensen, Apple Valley, MN (US); Julie A. Thompson, Circle Pines, MN (US)

(21) Appl. No.: 18/535,514

(22) Filed: Dec. 11, 2023

Related U.S. Application Data
(60) Provisional application No. 63/431,882, filed on Dec. 12, 2022.

(51) Int. Cl. A61M 5/172; A61B 5/00; A61B 5/021; A61B 7/00; A61N 1/365

(52) U.S. Cl. CPC A61M 5/1723; A61B 5/021; A61B 5/4836; A61B 5/686; A61B 5/7275; A61B 5/7282; A61B 7/00; A61N 1/36585; A61B 2562/0219

(57) ABSTRACT

Systems and methods are disclosed to determine a syncope metric for a patient as a function of cardiac acceleration information and the blood pressure information received from a signal receiver circuit and to determine an indication of a worsening risk of syncope or a syncope event for a patient using the determined syncope metric, wherein the determined indication of worsening risk of syncope or the syncope event can be provided to a user or a process.

### CLAIM OF PRIORITY

This application claims the benefit of U.S. Provisional Application No. 63/431,882, filed on Dec. 12, 2022, which is hereby incorporated by reference in its entirety.

### TECHNICAL FIELD

This document relates generally to medical devices and more particularly to a syncope prediction using cardiac acceleration information.

### BACKGROUND

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation. Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

Syncope is a temporary loss of consciousness caused by a fall in blood pressure, reducing oxygen supply to the brain. The most common type of syncope is neurally mediated syncope (NMS), where the part of the nervous system that regulates blood pressure and heart rate malfunctions in response to a trigger, such as emotional stress or pain. NMS typically occurs while standing, and is often preceded by a sensation of warmth, nausea, lightheadedness, tunnel vison, or visual disturbances. Placing the patient in a reclining position often restores blood flow and consciousness, ending the episode. Subcategories of NMS include situational syncope, often related to certain physical functions, such as coughing, laughing, or swallowing, carotid sinus syndrome (CSS), an abnormal response to carotid massage, and vasovagal syncope (VVS), a response to a sudden drop in blood flow to the brain.

Other types of syncope include orthostatic syncope, caused by a postural decrease in blood pressure that reduces blood flow to the cerebrum, and cardiac syncope, caused by various heart conditions including cardiac arrhythmias (e.g., bradycardia or tachycardia) or structural disorders, such as aortic stenosis, etc.

### SUMMARY

Systems and methods are disclosed to determine a syncope metric for a patient as a function of cardiac acceleration information and the blood pressure information received from a signal receiver circuit and to determine an indication of a worsening risk of syncope or a syncope event for a patient using the determined syncope metric, wherein the determined indication of worsening risk of syncope or the syncope event can be provided to a user or a process.

An example (e.g., “Example 1”) of subject matter (e.g., a medical device system) may comprise a signal receiver circuit configured to receive physiologic information of a patient, the physiologic information including cardiac acceleration information and blood pressure information of the patient, and an assessment circuit configured to determine a syncope metric for the patient as a function of cardiac acceleration information and the blood pressure information, determine an indication of a worsening risk of syncope or a syncope event for the patient using the determined syncope metric, and provide the determined indication of the worsening risk of syncope or the syncope event for the patient to a user or a process.

In Example 2, the subject matter of Example 1 may optionally be configured such that the cardiac acceleration information includes heart sound information of the patient, including second heart sound (S2) information, and the blood pressure information comprises an indication of systolic blood pressure.

In Example 3, the subject matter of any one or more of Examples 1-2 may optionally be configured such that the S2 information comprises a daily S2 value and the blood pressure information comprises a daily systolic blood pressure value.

In Example 4, the subject matter of any one or more of Examples 1-3 may optionally be configured such that the assessment circuit is configured to determine the syncope metric using an increase in the cardiac acceleration information and a decrease in the blood pressure information and the assessment circuit is configured to determine the indication of the worsening risk of syncope or the syncope event for the patient using a comparison of the determined syncope metric to a threshold.

In Example 5, the subject matter of any one or more of Examples 1˜4 may optionally be configured such that the assessment circuit is configured to determine a baseline syncope metric using physiologic information of the patient and the assessment circuit is configured to determine the indication of the worsening risk of syncope or the syncope event for the patient using a comparison of the determined syncope metric to the baseline syncope metric.

In Example 6, the subject matter of any one or more of Examples 1-5 may optionally be configured to include an ambulatory medical device including an accelerometer configured to sense the cardiac acceleration information and an external medical device including a pressure sensor configured to sense a systolic blood pressure of the patient, wherein the blood pressure information comprises the sensed systolic blood pressure information, the signal receiver circuit is configured to receive the cardiac acceleration information from the ambulatory medical device and the systolic blood pressure information from the external medical device, and the assessment circuit is configured to provide a control to at least one of the ambulatory medical device or the external medical device using the determined indication of the worsening risk of syncope or the syncope event for the patient.

In Example 7, the subject matter of any one or more of Examples 1-6 may optionally be configured such that the assessment circuit is configured to transition a medical device of the medical device system from a first low-power mode to a second high-power mode using the determined risk of syncope or the syncope event for the patient.

In Example 8, the subject matter of any one or more of Examples 1-7 may optionally be configured such that the first low-power mode comprises an ambulatory monitoring mode, the second high-power mode comprises a higher-power monitoring mode including storing received cardiac acceleration information and the received blood pressure information in long-term storage, and the medical device comprises an implantable medical device comprising the signal receiver circuit and the assessment circuit.

In Example 9, the subject matter of any one or more of Examples 1-8 may optionally be configured such that the assessment circuit is configured to determine the indication of the worsening risk of syncope or the syncope event for the patient using an increase in the cardiac acceleration information of the patient greater than a first threshold and a decrease in the blood pressure information of the patient greater than a second threshold.

In Example 10, the subject matter of any one or more of Examples 1-9 may optionally be configured such that the assessment circuit is configured to detect a perturbation event using the received physiologic information or receiving an indication of the perturbation event, the assessment circuit is configured to determine the syncope metric for the patient using received cardiac acceleration information occurring over an early window of the detected perturbation event, and the early window follows the detected perturbation event after a first blanking period.

In Example 11, the subject matter of any one or more of Examples 1-10 may optionally be configured such that to provide the determined indication of the worsening risk of syncope or the syncope event for the patient to the user or the process comprises to provide a trigger to update the received blood pressure information in response to the determined indication of the worsening risk of syncope or the syncope event.

In Example 12, the subject matter of any one or more of Examples 1-11 may optionally be configured such that the signal receiver circuit is configured to receive the cardiac acceleration information of the patient, including heart sound information of the patient, including at least two of: first heart sound (S1) information, second heart sound (S2) information, third heart sound (S3) information, and fourth heart sound (S4) information, and, in response to the determined indication of the worsening risk of syncope or the syncope event for the patient, the assessment circuit is configured to determine a predicted fainting metric for the patient using the at least two of the first S1 information, the S2 information, the S3 information, and the S4 information.

In Example 13, the subject matter of any one or more of Examples 1-12 may optionally be configured to include a therapy circuit configured to alter or provide one of a drug therapy or a pacing therapy in response to the determined indication of the worsening risk of syncope or the syncope event.

An example (e.g., “Example 14”) of subject matter (e.g., a method) may comprise receiving physiologic information of a patient using a signal receiver circuit, the physiologic information including cardiac acceleration information and blood pressure information of the patient, and, using an assessment circuit, determining a syncope metric for the patient as a function of cardiac acceleration information and the blood pressure information, determining an indication of a worsening risk of syncope or a syncope event for the patient using the determined syncope metric, and providing the determined indication of the worsening risk of syncope or the syncope event for the patient to a user or a process.

In Example 15, the subject matter of any one or more of Examples 1-14 may optionally be configured such that the cardiac acceleration information includes heart sound information of the patient, including second heart sound (S2) information and the blood pressure information comprises an indication of systolic blood pressure.

In Example 16, the subject matter of any one or more of Examples 1-15 may optionally be configured such that the S2 information comprises a daily S2 value and the blood pressure information comprises a daily systolic blood pressure value.

In Example 17, the subject matter of any one or more of Examples 1-16 may optionally be configured such that determining the syncope metric comprises using an increase in the cardiac acceleration information and a decrease in the blood pressure information and determining the indication of the worsening risk of syncope or the syncope event for the patient comprises using a comparison of the determined syncope metric to a threshold.

In Example 18, the subject matter of any one or more of Examples 1-17 may optionally be configured to include determining, using the assessment circuit, a baseline syncope metric using physiologic information of the patient, wherein determining the indication of the worsening risk of syncope or the syncope event for the patient comprises using a comparison of the determined syncope metric to the baseline syncope metric.

In Example 19, the subject matter of any one or more of Examples 1-18 may optionally be configured to include sensing the cardiac acceleration information using an accelerometer of an ambulatory medical device, sensing a systolic blood pressure of the patient using a pressure sensor of an external medical device, wherein the blood pressure information comprises the sensed systolic blood pressure information, and providing, using the assessment circuit, a control to at least one of the ambulatory medical device or the external medical device using the determined indication of the worsening risk of syncope or the syncope event for the patient, wherein receiving the cardiac acceleration information comprises from the ambulatory medical device and receiving the systolic blood pressure information comprises from the external medical device.

In Example 20, the subject matter of any one or more of Examples 1-19 may optionally be configured to include transitioning a medical device, using the assessment circuit, from a first low-power mode to a second high-power mode using the determined risk of syncope or the syncope event for the patient, wherein the first low-power mode comprises an ambulatory monitoring mode and the second high-power mode comprises a higher-power monitoring mode including storing received cardiac acceleration information and the received blood pressure information in long-term storage.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals

may describe similar components in different views. Like numerals having different letter suffixes may represent different instances of similar components. The drawings illustrate generally, by way of example, but not by way of limitation, various embodiments discussed in the present document.

FIG. 1 illustrates a relationship between measured patient physiologic information including patient heart sound information.

FIGS. 2A-2B illustrate example relationships of an S2 parameter for different patient groups.

FIG. 3 illustrates an example relationship of systolic blood pressure for different patient groups.

FIGS. 4A-4B illustrate example relationships of an S3 parameter for different patient groups.

FIGS. 5A-5B illustrate example relationships of an S4 parameter for different patient groups.

FIGS. 6A-6B illustrate example first and second reflex syncope detections using different physiologic information.

FIGS. 7A-7B illustrate example first and second fainting predictions using different physiologic information during a perturbation.

FIG. 8 illustrates an example method of determining an indication of a worsening risk of syncope or a syncope event for a patient.

FIG. 9 illustrates an example system (e.g., a medical device system).

FIG. 10 illustrates an example patient management system and portions of an environment in which the patient management system may operate.

FIG. 11 illustrates a block diagram of an example machine upon which any one or more of the techniques discussed herein may perform.

### DETAILED DESCRIPTION

Ambulatory medical devices can include, or be configured to receive cardiac electrical information from, one or more electrodes located within, on, or proximate to the heart, such as coupled to a lead and located in one or more chambers of the heart or within the vasculature of the heart near one or more chambers. Ambulatory medical devices can additionally include or be configured to receive mechanical acceleration information from one or more accelerometer sensors to determine and monitor patient acceleration information, such as cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc.

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow.

Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

The present inventors have recognized, among other things, systems and methods to determine or predict a risk or increased risk of syncope or a syncope event (e.g., fainting) using cardiac acceleration information (e.g., heart sound information, etc.), such as during a perturbation. As described herein, perturbations generally include a tilt table test, but in other examples can include real world changes in position, posture, certain activities, etc. The systems and methods disclosed herein can determine or predict the risk of syncope or the syncope event earlier and with a higher sensitivity and specificity than traditional measures, such as those based on heart rate or blood pressure, providing additional and improved functionality to existing sensor systems.

In certain examples, earlier detection of increased risk of syncope or the syncope event can enable existing medical devices and medical device systems to more accurately alter or transition between different sensing or therapy modes or otherwise change settings or parameters earlier to detect and capture valuable physiologic information leading up to and during a syncope event. For example, additional sensing (e.g., blood pressure sensing, increased time periods of cardiac acceleration sensing, etc.) can be triggered, further improving detection and diagnosis, or at least making additional information available for clinician review. In other examples, sensing modes can be changed (e.g., from a low-power mode to a high-power mode, altering periods of detection of certain parameters, etc.), time periods of detected or determined parameters can be increased (e.g., detecting changes over longer time periods, such as multiple minutes, instead of seconds, etc.), sampling frequency or resolution of detected, sensed, or sampled information can be increased, data storage periods can be increased, or one or more notifications, alerts, therapies, or therapy parameters can be provided or changed based on the detected indication. Further, the detected indication can be used as an additional measure to improve existing syncope detection, such as using blood pressure and heart rate based measures, improving the sensitivity, specificity, or confidence of detected patient conditions, medical event storage, transmission, alerts, etc.

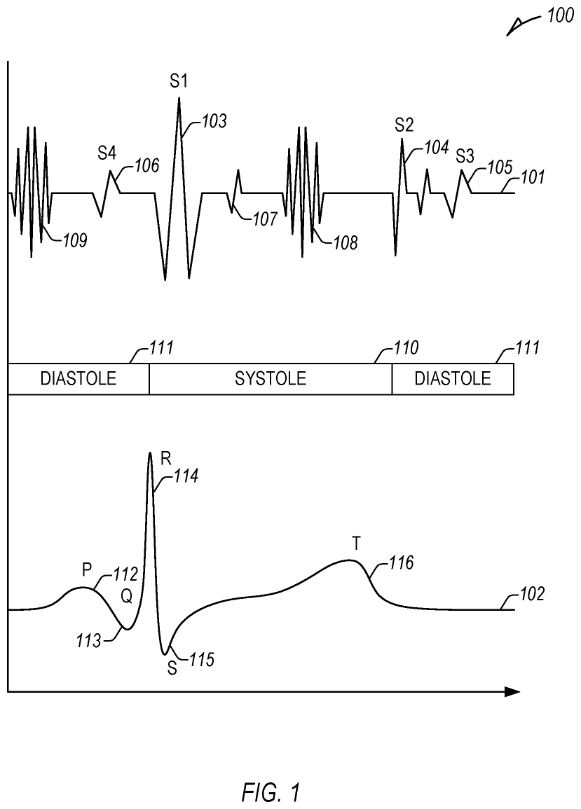

FIG. 1 illustrates a relationship 100 between measured patient physiologic information including patient heart sound information 101 (including a first heart sound (S1) 103, a second heart sound (S2) 104, a third heart sound (S3) 105, a fourth heart sound (S4) 106, ejection sounds 107, systolic murmurs 108, opening sounds between S2 and S3, and diastolic murmurs 109) and cardiac electrical information 102 (including a P wave 112, a Q wave 113, an R wave 114, an S wave 115, and a T wave 116 of an electrocardiogram signal) over a cardiac cycle, including periods of systole 110 and diastole 111.

The horizontal axis in FIG. 1 is time, and is shown without scale, as the time of the cardiac cycle depends upon the heart rate of the patient, which greatly varies (e.g., typically between 60 and 100 beats per minute (bpm), but more or less in certain examples). Systole 110 generally starts at the R wave 114 and the occurrence of S1 103 and ending at the T wave 116. Diastole 111 generally starts after the T wave 116 and at the occurrence of S2 104 and includes S3 105 and S4 106. The duration of diastole 111 is typically longer than the duration of systole 110, frequently by a factor of 2, etc.

Forceful ventricular contraction in an unmet chamber resulting from, in certain examples, reduced ventricular filling or a relaxation of the ventricle coupled with a sympathetic trigger, can result in a syncope event. One driver of the S2 is a pressure difference between the aorta and the left ventricle at the time of the aortic valve closure (e.g., a pressure gradient, aortic pressure minus left ventricular pressure, at the dicrotic notch). Accordingly, the S2 response can be used to determine an indication of a syncope event, such as described in the commonly assigned Shute et al. U.S. Application Ser. No. 63/302,432 entitled “Heart Sound Based Syncope Detection,” which is hereby incorporated by reference in its entirety, including its disclosure of detecting a potential syncope event using cardiac acceleration information of the patient.

The present inventors have recognized, among other things, that the baseline S2 value itself (or a deviation from the baseline S2 value), separate from a perturbation, can be used to determine an indication of a risk of syncope, to identify likely fainters or patients at a risk of fainting, or to distinguish likely fainters from non-fainters during a perturbation.

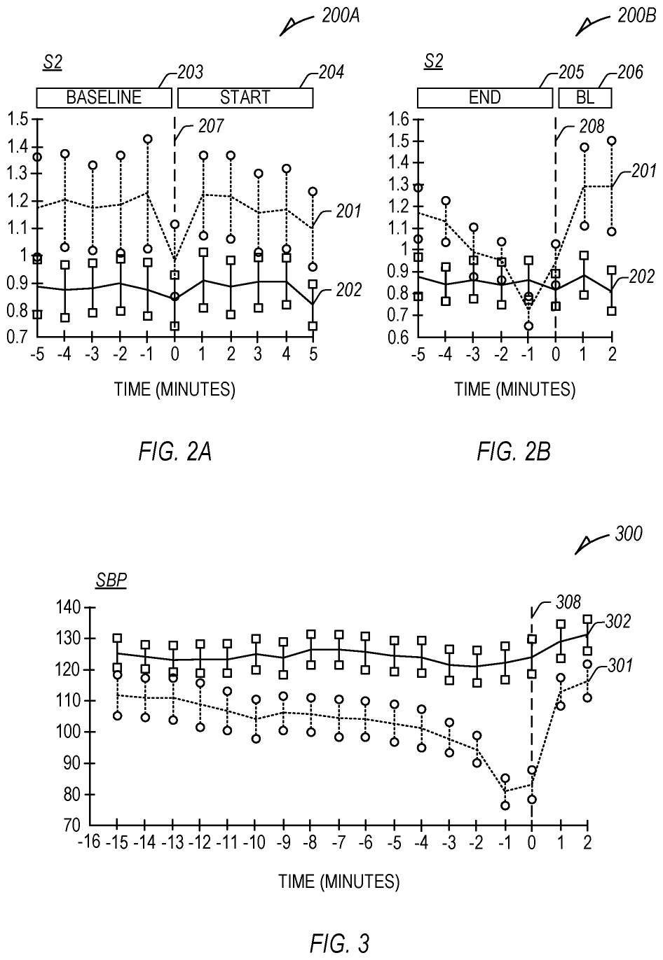

FIGS. 2A-2B illustrate example relationships 200A, 200B of an S2 parameter for different patient groups before, during, and after a perturbation event. The patient groups can include a fainters group illustrated with a first S2 trend 201 in a dashed line with group variance bound by circles and a non-fainters group illustrated with a second S2 trend 202 in a solid line with group variance bound by squares.

The fainters group can include patients diagnosed as fainting or at a high risk of fainting during the perturbation event. The non-fainters group can include a control group, patients diagnosed as a non-fainter, or patients determined to have a low risk of fainting during a perturbation event. The S2 parameters can include, in certain examples, an S2 energy value (e.g., an S2 RMS value) or one or more other individual or composite S2 values, such as an S2 amplitude, a change in an S2 parameter, or an S2 parameter normalized by one or more other parameters, etc.

First and second baseline (BL) periods 203, 206 precede and follow a perturbation start 207 and a perturbation end 208, respectively, on either side of a perturbation start period 204 and a perturbation end period 205. The perturbation start period 204 includes the first several minutes (e.g., 5 minutes, etc.) of the perturbation event. The perturbation end period 205 includes the last several minutes (e.g., 5 minutes) of the perturbation event. The times along the horizontal axes are in minutes with respect to the perturbation start 207 and the perturbation end 208, respectively.

First, the baseline difference between the first and second S2 trends 201, 202 is large, with the fainters group having a baseline S2 parameter more than 30% higher than the non-fainters group, indicating that a S2 value, alone or with respect to one or more other measures, can be used to determine an increases risk of syncope. Additionally, an increase in S2 generally over time can be indicative of an increasing risk of the patient having syncope or a future a syncope event.

Second, while the second S2 trend 202 is relatively stable throughout the example relationships 200A, 200B, the first S2 trend 201 illustrates comparatively large changes both during the perturbation start period 204 (e.g., a more substantial deviation from an established baseline at the perturbation start 207 than the second S2 trend 202, etc.) and during the perturbation end period 205 (e.g., a substantial deviation and downward trend before the perturbation end 208 and subsequent recovery into the second baseline period 206). The first S2 trend 201 returns to its first baseline period 203 value after the perturbation end 208 (which confirms a systematic difference between fainters and non-fainters that is not related to anticipation of the perturbation, which may be associated with the drop and subsequent rise about the perturbation start 207).

FIG. 3 illustrates an example relationship 300 of systolic blood pressure for different patient groups during and after a perturbation event ending at a perturbation end 308. The different patient groups include a fainters group illustrated with a first systolic blood pressure (SBP) trend 301 in a dashed line with group variance bound by circles and a non-fainters group illustrated with a second systolic blood pressure trend 302 in a solid line with group variance bound by squares.

The first systolic blood pressure trend 301 shows a drop in systolic blood pressure more than 20 mm Hg toward the end of the perturbation event. The second systolic blood pressure trend 302 shows little movement during the perturbation event. The difference in systolic blood pressure between the different patient groups is relatively large, with the first systolic blood pressure trend 301 having a baseline value more than 10% higher than the systolic blood pressure trend 302.

The present inventors have recognized, among other things, that an indication of one or both of lower or decreasing pressure in the left ventricle or lower volume in the left ventricle at the time of activation can be determined as a function of a relatively high S2 parameter (or an increase in an S2 parameter over a time period), in certain examples, coupled with a relatively low systolic blood pressure (or a decrease in systolic blood pressure over the time period). The determined indication can be used to detect a relatively low, lower, or decreasing pressure in the left ventricle or a lower volume in the left ventricle at the time of activation, resulting in an increased risk of an activation with an unmet chamber, and accordingly, an increased risk of fainting.

For example, the S3 and S4 are related to filling pressures of the left ventricle during diastole. The S3 occurs in the early-diastolic period and can be indicative of an abrupt or early halt in diastolic filling. The S4 occurs in the late-diastolic period before the R wave or activation of the left ventricle and is related to late ventricular filling. A reduction in early or late ventricular filling can be indicative of a reduced left ventricular end-diastolic pressure, and coupled with hypercontractility, such as resulting from one or more trigger events (e.g., emotional stress, pain, etc.), can indicate an increased risk of a syncope event.

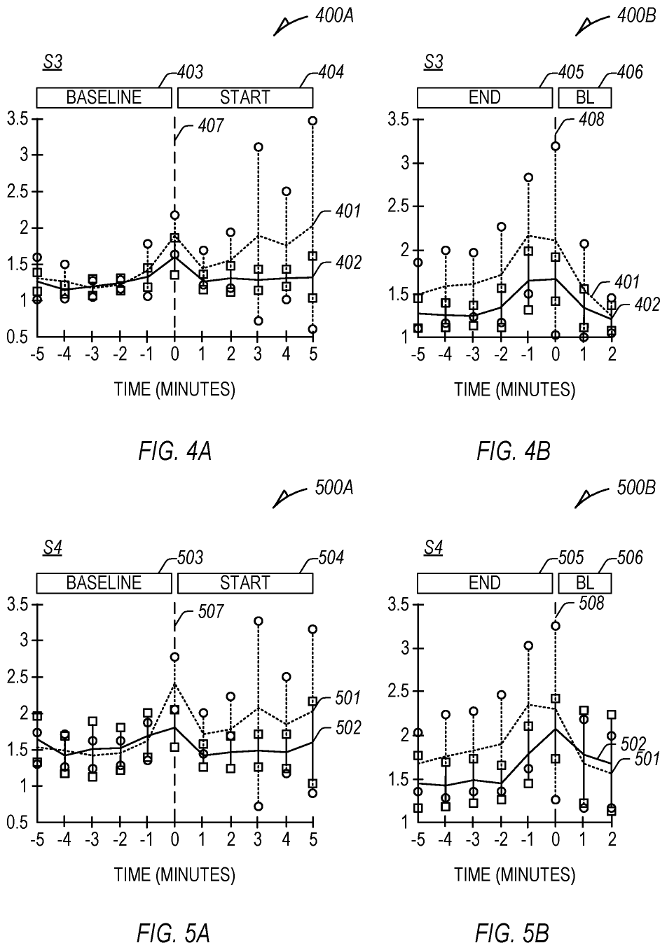

FIGS. 4A-4B illustrate example relationships 400A, 400B of an S3 parameter for different patient groups before, during, and after a perturbation event. The patient groups can include a reflex syncope group having a first S3 trend 401 in a dashed line with group variance bound by circles and a non-reflex syncope group illustrated having a second S3 trend 402 in a solid line with group variance bound by squares.

The reflex syncope patients can include patients diagnosed as having reflex syncope, fainting, or to have a high risk of fainting during the perturbation event. The non-reflex syncope group can include a control group or patients diagnosed or determined as not having reflex syncope. The S3 parameters can include, in certain examples, an S3 amplitude or energy value or one or more other individual or composite S3 values, such as an S3 amplitude, a change in an S3 parameter, or an S2 parameter normalized by one or more other parameters, etc. (e.g., an S3 ensemble parameter or one or more other individual or composite S3 values, such as an S3 amplitude or energy, etc.).

First and second baseline (BL) periods 403, 406 precede and follow a perturbation start 407 and a perturbation end 408, respectively, on either side of a perturbation start period 404 and a perturbation end period 405. The perturbation start period 404 includes the first several minutes (e.g., 5 minutes, etc.) of the perturbation event. The perturbation end period 405 includes the last several minutes (e.g., 5 minutes) of the perturbation event. The times along the horizontal axes are in minutes with respect to the perturbation start 407 and the perturbation end 408, respectively.

FIGS. 5A-5B illustrate example relationships 500A, 500B of an S4 parameter for different patient groups before, during, and after a perturbation event. The patient groups can include a reflex syncope group having a first S4 trend 501 in a dashed line with group variance bound by circles and a non-reflex syncope group illustrated having a second S4 trend 502 in a solid line with group variance bound by squares.

The S3 parameters can include, in certain examples, an S3 amplitude or energy value or one or more other individual or composite S3 values, such as an S3 amplitude, a change in an S3 parameter, or an S2 parameter normalized by one or more other parameters, etc. (e.g., an S3 ensemble parameter or one or more other individual or composite S3 values, such as an S3 amplitude or energy, etc.).

First and second baseline (BL) periods 503, 506 precede and follow a perturbation start 507 and a perturbation end 508, respectively, on either side of a perturbation start period 504 and a perturbation end period 505. The perturbation start period 504 includes the first several minutes (e.g., 5 minutes, etc.) of the perturbation event. The perturbation end period 505 includes the last several minutes (e.g., 5 minutes) of the perturbation event. The times along the horizontal axes are in minutes with respect to the perturbation start 507 and the perturbation end 508, respectively.

The present inventors have recognized, among other things, that early S3 and S4 parameter features during a perturbation can be used to stratify the different reflex syncope and non-reflex syncope patient groups, such as without undergoing a full perturbation test, such as a tilt table test having different durations, commonly 10, 15, or 20 minutes or more, etc.

In certain examples, differences between the groups in FIGS. 4A and 4B and FIGS. 5A and 5B can be used to stratify patients into a respective groups. For example, for each of the relationships 400, 500, the S3 and S4 parameters were similar during the baseline periods before and after perturbations, but varied during the perturbation start periods 404, 504. In FIG. 4A, the first S3 trend 401 rises faster over the perturbation start period 404 than the second S3 trend 402 over the same period. In certain examples, this variance is especially great in an early time period after a first blanking period (e.g., 1 minute, 2 minutes, etc.) after the perturbation start 407, such that the early time period is between minutes 2 and 5 of the perturbation start period 404 or one or more other early time periods after the first blanking period (e.g., between minutes 1 and 5, between minutes 2 and 6, between minutes 3 and 5, etc.).

Additionally, whereas both the first and second S3 trends 401, 402 rise at (and possibly in anticipation of) the perturbation start 407, the second S3 trend 402 falls substantially back to a baseline level (e.g., a value of the baseline period 403), whereas the first S3 trend 401 remains elevated above its corresponding baseline level. Accordingly, a difference between a metric indicative of the value, a slope, or a combination thereof of the first S3 trend 401 in the early time period (e.g., after the first blanking period) and a metric indicative of a corresponding baseline value (e.g., in a corresponding period prior to the perturbation start 407, etc.) can be used to distinguish the different groups.

FIG. 5A illustrates similar response with respect to the first and second S4 trends 501, 502, with the second S4 trend 502 falling substantially back to a baseline level, whereas the first S4 trend 501 remains elevated above its corresponding baseline level.

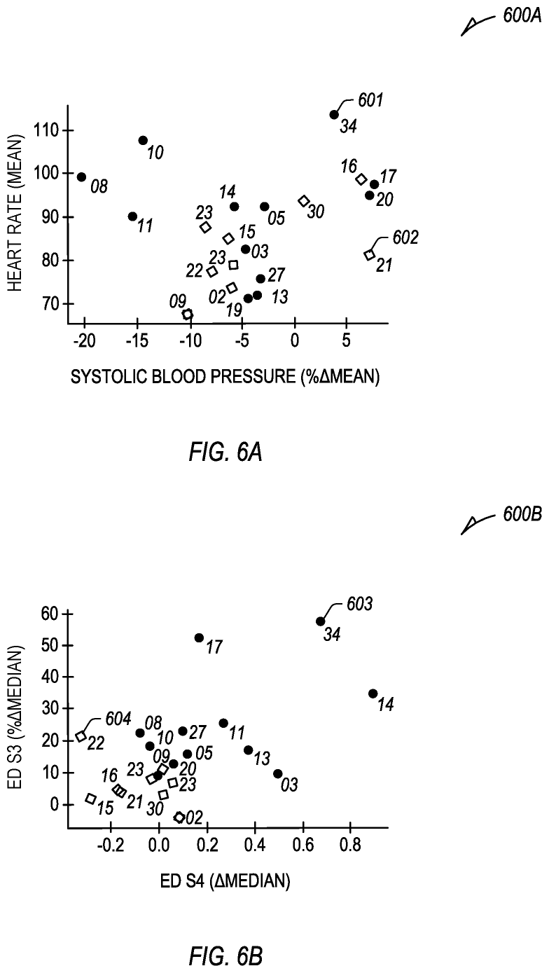

FIGS. 6A-6B illustrate example first and second reflex syncope detections 600A, 600B using different physiologic information during a perturbation. FIG. 6A illustrates a first example detection 600A using heart rate information and systolic blood pressure across a set of patients (numbered), including patients having a diagnosis of reflex syncope illustrated with dark circles (e.g., circle 601, representing patient 34 in FIG. 6A) and patients having a non-reflex syncope diagnosis (e.g., a normal or control group) illustrated with squares (e.g., square 602, representing patient 21 in FIG. 6A). The set of patients in FIG. 6A are largely scattered.

In contrast, FIG. 6B illustrates a second example detection 600B using S3 and S4 information across the same set of patients (numbered), including patients having a diagnosis of reflex syncope illustrated with dark circles (e.g., circle 603, representing patient 34 in FIG. 6B) and patients having a non-reflex syncope diagnosis (e.g., a normal or control group) illustrated with squares (e.g., square 604, representing patient 22 in FIG. 6B).

Accordingly, the present inventors have recognized that heart sound features, including one or more of S3 or S4 features or combinations thereof, such as in an early time period following a perturbation (e.g., in contrast to a baseline, in certain examples following a blanking period, etc.), can be used to detect the presence of an underlying syncope risk, and that such detection is more sensitive and specific than traditional heart rate and blood pressure measures. Although illustrated with respect to different measures of S3 and S4 in FIG. 6B (e.g., an ensemble deviation (ED) S3 (% Δmedian) and ED S4 (Δmedian), respectively), in other examples one or more other S3 or S4 measures or parameters or combinations thereof can be used. Ensemble deviations can include beat-to-beat variability of heart sound information collected over a number of beats (e.g., 16 or 32 successive beats, etc.). In contrast, ensemble averages can include an average of heart sound information collected over a number of beats (e.g., 16 or 32 successive beats, etc.). Composite heart sound information can include, in certain examples, an ensemble deviation, an ensemble average, or one or more other combined metrics information from one or more heart sounds, etc.

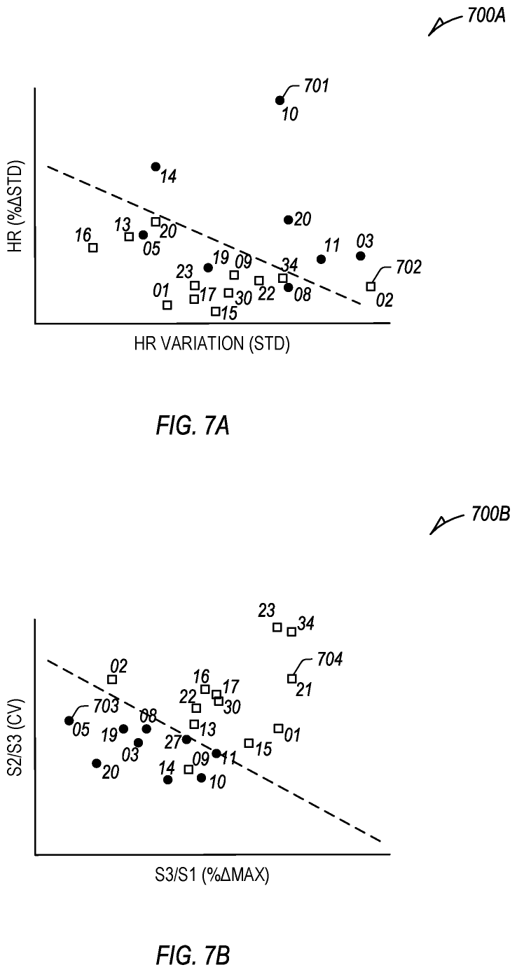

FIGS. 7A-7B illustrate example first and second fainting predictions 700A, 700B using different physiologic information during a perturbation. FIG. 7A illustrates a first example prediction 700A using heart rate information (e.g., a comparison of heart rate (% Δstd) and heart rate variation (std), etc.) across a set of patients (numbered), including patients having a diagnosis of fainting, such as during a perturbation, illustrated with dark circles (e.g., circle 701, representing patient 10 in FIG. 7A) and patients having a non-fainting diagnosis (e.g., a normal or control group) illustrated with squares (e.g., square 702, representing patient 02 in FIG. 7A).

In contrast, FIG. 7B illustrates a second example prediction 700B using combinations of composite heart sound metrics across the same set of patients (numbered), including patients having a diagnosis of fainting illustrated with dark circles (e.g., circle 703, representing patient 05 in FIG. 7B) and patients having a non-fainting diagnosis (e.g., a normal or control group) illustrated with squares (e.g., square 704, representing patient 21 in FIG. 7B).

Accordingly, the present inventors have recognized that heart sound features, including one or more of S1, S2, S3, or S4 features or combinations thereof, such as in an early time period following a perturbation (e.g., in contrast to a baseline, in certain examples following a blanking period, etc.), can be used to determine a fainting prediction indication, and that such detection is more sensitive and specific than traditional heart rate measures. Although illustrated with respect to different measures of S1, S2, and S3 in FIG. 7B (e.g., a ratio of S2/S3 (cv) and S3/S1 (% Amax), respectively), in other examples one or more other heart sounds measures or parameters or combinations thereof can be used.

Similar features detected in conjunction with one or more changes can be used to determine a risk of experiencing a future syncope event, such as within a time period, or otherwise determine a measure or risk of possible syncope event, such as to determine a worsening patient condition, etc.

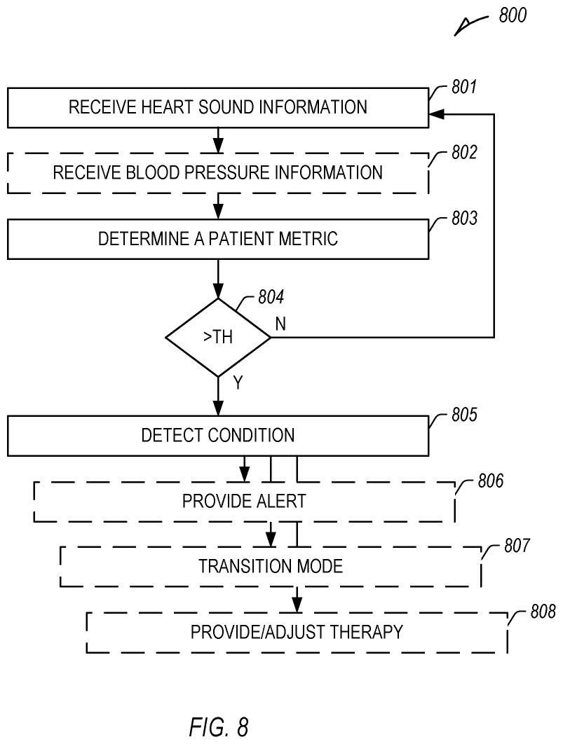

FIG. 8 illustrates an example method 800 of determining an indication of a worsening risk of syncope or a syncope event for a patient using a syncope metric determined as a function of cardiac acceleration information, such as using an assessment circuit of a medical device system (e.g., an implantable medical device, an ambulatory medical device, etc.).

At step 801, heart sound information or other cardiac acceleration information can be received, such as using a signal receiver circuit of the medical device system, from a heart sound sensor (e.g., an accelerometer, etc.), or a heart sound sensor circuit.

At step 802, blood pressure information can be optionally received, such as using the signal receiver circuit of the medical device system, from a blood pressure sensor (e.g., an external blood pressure cuff, a finger worn blood pressure sensor, an implantable pressure sensor, etc.) or a blood pressure processing circuit coupled to the blood pressure sensor. In other examples, other physiologic information can be received, such as one or more of cardiac electrical information (e.g., heart rate information, etc.), activity information of the patient, or posture information of the patient from one or more other sensor or sensor circuits, etc.

At step 803, a patient metric can be determined, such as a syncope metric for the patient, using an assessment circuit and physiologic information from the signal receiver circuit, such as cardiac acceleration information, etc.

In certain examples, the syncope metric can be determined as a measure or value of at least one heart sound parameters, including one or more of S1, S2, S3, or S4 (e.g., a daily value, an amplitude or energy value (e.g., an energy value in a heart sound window defined by, among other things, a cardiac signal feature, one or more other heart sounds, or combinations thereof, over one or more cardiac cycles, etc.), etc. In other examples, the syncope metric can be determined as a function of cardiac acceleration information and blood pressure information (e.g., daily values, weekly values, ensemble average values, etc.). In an example, the cardiac acceleration information can include S2 information, and the blood pressure information can include an indication of systolic blood pressure of the patient. An example equation, equation (1), is provided below.

p(Syncope)∝(α×S2)/(B×SBP) (1)

In equation (1), p(Syncope) is a syncope metric, S2 is a measure of patient heart rate, SBP is a measure of patient systolic blood pressure, and a and B are variables. One indication of a worsening risk of syncope or a syncope event can be determined with respect to the relative direction of the multiple components. For example, an increasing S2 and a decreasing SBP can be indicative of a worsening risk of syncope or a syncope event. In other examples, other functions or equations can be used.

At step 804, in an example, the syncope metric can be determined, then compared to one or more thresholds (TH) or baselines to determine the indication for the patient. In an example, one or more of the baselines or thresholds can be determined by the assessment circuit. In an example, the one or more thresholds, parameters, or orders of parameters can be relative parameters, such as relative to a baseline value, leading up to the syncope or potential syncope event, or as otherwise illustrated in FIGS. 1-7B. If the syncope metric is greater than the threshold, at step 805, a condition can be detected, and at step 806, an alarm, alert, or a notification can optionally be provided, such as to a user or process.

In certain examples, the alarm, alert, or notification can optionally be provided to the patient or a caregiver of the patient and can include a notification that the risk of a syncope event is high or worsening. In certain examples, a notification to eat some salty food, sit down, stay hydrated (e.g., drink fluids), or see a doctor can accompany the notification.

At step 807, a mode can be transitioned based on the detected condition. In certain examples, a therapy can be provided in response to the detected condition. For example, the assessment circuit can be transitioned from a medical device having a first monitoring mode (e.g., a low-power mode) to a second, active monitoring mode. In certain examples, one or more settings can be changed, such as accelerometer sensitivity to pick up potential triggers (e.g., prolonged periods of upright standing, abrupt changes in posture, abrupt changes in heart rate, etc.). In contrast to a monitoring mode where such measures are taken intermittently, a detected increase in the determined syncope metric can transition or trigger implementation of certain features. In other examples, heart sound sampling can occur more frequently, and when the heart sound information increases, one or more additional corresponding measurements can be triggered, such as an external blood pressure measurement, etc.

At step 808, a therapy can be adjusted or provided. For example, a pacing therapy (e.g., DDD pacing) can be provided, enabled, or adjusted, such as to reduce syncope due to a reduction in heart rate, ventricular filling, or blood pressure associated with a syncope event, for example, such as disclosed in the commonly assigned Sanders U.S. Pat. No. 8,761,878 entitled “IMPLANTABLE CARDIAC MONITOR UPGRADEABLE TO PACEMAKER OR CARDIAC RESYNCHORNIZATION DEVICE,” or in the commonly assigned Spinelli et al. U.S. Pat. No. 8,498,703 entitled “METHOD AND SYSTEM FOR TREATMENT OF NEUROCARDIOGENIC SYNCOPE,” each of which are hereby incorporated by reference in their entireties, including their disclosure of providing a pacing therapy or pacing pulses to alleviate or at least partially avoid syncope.

At step 808, a vasoconstrictor (e.g., pressor drugs, etc.) can be triggered, provided, or adjusted, such as using a drug pump, in response to the detected condition, to contract or constrict the blood vessels, counteracting hypotension, a pooling of blood in the extremities, or a lack of blood supply to the brain, such as to alleviate or at least partially avoid syncope. In other examples, application of a vasoconstrictor can be triggered in response to the detected condition, alone or in combination with a pacing therapy, such as that described above, such as to both increase arterial pressure and to maintain cardiac output.

At step 808, in certain examples, the detected condition can be used to trigger one or more additional measurements, such as a cuff-based blood pressure measurement, in certain examples based in part on the received cardiac acceleration information. In certain examples, the blood pressure measurements can lag the cardiac acceleration measurements, as certain blood pressure cuffs take time to make readings. In contrast, the cardiac acceleration measurements can be done using an implantable, wearable, or ambulatory medical device without requiring additional triggers, etc. In other examples, a blood pressure measurement can trigger cardiac acceleration or other physiologic information.

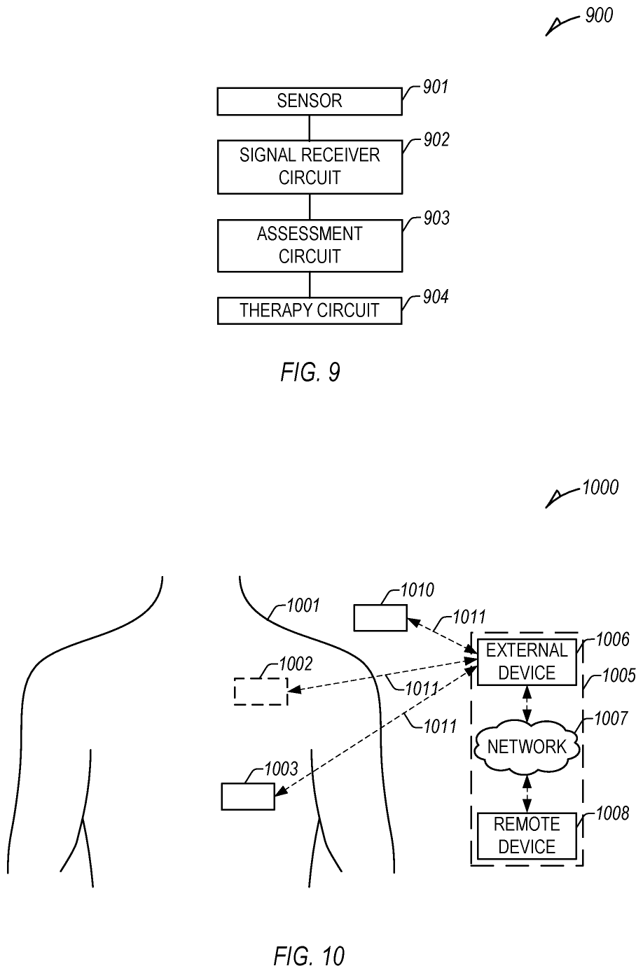

FIG. 9 illustrates an example system 900 (e.g., a medical device system). In an example, one or more aspects of the example system 900 can be a component of, or communicatively coupled to, a medical device, such as an implantable medical device (IMD), an insertable cardiac monitor, an ambulatory medical device (AMD), etc. The system 900 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 900 can include a single medical device or a plurality of medical devices implanted in a patient's body or otherwise positioned on or about the patient to monitor patient physiologic information of the patient using one or more sensors, such as a sensor 901. In an example, the sensor 901 can include one or more of: a respiration sensor configured to receive respiration information (e.g., a respiration rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 900 can include a signal receiver circuit 902 and an assessment circuit 903. The signal receiver circuit 902 can be configured to receive physiologic information of a patient (or group of patients) from the sensor 901. The assessment circuit 903 can be configured to receive information from the signal receiver circuit 902, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 903 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The assessment circuit 903 can be configured to provide an output to a user, such as to a display or one or more other user interface, the output including a score, a trend, an alert, or other indication. In other examples, the assessment circuit 903 can be configured to provide an output to another circuit, machine, or process, such as a therapy circuit 904 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 904 can include one or more of a stimulation control circuit, a cardiac stimulation circuit, a neural stimulation circuit, a dosage determination or control circuit, etc. In other examples, the therapy circuit 904 can be controlled by the assessment circuit 903, or one or more other circuits, etc.

FIG. 10 illustrates an example patient management system 1000 and portions of an environment in which the patient management system 1000 may operate. The patient management system 1000 can perform a range of activities, including remote patient monitoring and diagnosis of a disease condition. Such activities can be performed proximal to a patient 1001, such as in a patient home or office, through a centralized server, such as in a hospital, clinic, or physician office, or through a remote workstation, such as a secure wireless mobile computing device.

The patient management system 1000 can include one or more medical devices, an external system 1005, and a communication link 1011 providing for communication between the one or more ambulatory medical devices and the external system 1005. The one or more medical devices can include an ambulatory medical device (AMD), such as an implantable medical device (IMD) 1002, a wearable medical device 1003, or one or more other implantable, leadless, subcutaneous, external, wearable, or medical devices configured to monitor, sense, or detect information from, determine physiologic information about, or provide one or more therapies to treat various conditions of the patient 1001, such as one or more cardiac or non-cardiac conditions (e.g., dehydration, sleep disordered breathing, etc.).

In an example, the implantable medical device 1002 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 1001. In another example, the implantable medical device 1002 can include a monitor implanted, for example, subcutaneously in the chest of patient 1001, the implantable medical device 1002 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 1002 can include an assessment circuit configured to detect or determine specific physiologic information of the patient 1001, or to determine one or more conditions or provide information or an alert to a user, such as the patient 1001 (e.g., a patient), a clinician, or one or more other caregivers or processes, such as described herein. The implantable medical device 1002 can alternatively or additionally be configured as a therapeutic device configured to treat one or more medical conditions of the patient 1001. The therapy can be delivered to the patient 1001 via the lead system and associated electrodes or using one or more other delivery mechanisms. The therapy can include delivery of one or more drugs to the patient 1001, such as using the implantable medical device 1002 or one or more of the other ambulatory medical devices, etc. In some examples, therapy can include CRT for rectifying dyssynchrony and improving cardiac function in heart failure patients. In other examples, the implantable medical device 1002 can include a drug delivery system, such as a drug infusion pump to deliver drugs to the patient for managing arrhythmias or complications from arrhythmias, hypertension, hypotension, or one or more other physiologic conditions. In other examples, the implantable medical device 1002 can include one or more electrodes configured to stimulate the nervous system of the patient or to provide stimulation to the muscles of the patient airway, etc.

The wearable medical device 1003 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 1005 can include a dedicated hardware/software system, such as a programmer, a remote server-based patient management system, or alternatively a system defined predominantly by software running on a standard personal computer. The external system 1005 can manage the patient 1001 through the implantable medical device 1002 or one or more other ambulatory medical devices connected to the external system 1005 via a communication link 1011. In other examples, the implantable medical device 1002 can be connected to the wearable medical device 1003, or the wearable medical device 1003 can be connected to the external system 1005, via the communication link 1011. This can include, for example, programming the implantable medical device 1002 to perform one or more of acquiring physiologic data, performing at least one self-diagnostic test (such as for a device operational status), analyzing the physiologic data, or optionally delivering or adjusting a therapy for the patient 1001. Additionally, the external system 1005 can send information to, or receive information from, the implantable medical device 1002 or the wearable medical device 1003 via the communication link 1011. Examples of the information can include real-time or stored physiologic data from the patient 1001, diagnostic data, such as detection of patient hydration status, hospitalizations, responses to therapies delivered to the patient 1001, or device operational status of the implantable medical device 1002 or the wearable medical device 1003 (e.g., battery status, lead impedance, etc.). The communication link 1011 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 1005 can include an external device 1006 in proximity of the one or more ambulatory medical devices, and a remote device 1008 in a location relatively distant from the one or more ambulatory medical devices, in communication with the external device 1006 via a communication network 1007. Examples of the external device 1006 can include a medical device programmer. The remote device 1008 can be configured to evaluate collected patient or patient information and provide alert notifications, among other possible functions. In an example, the remote device 1008 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 1008 can receive data from multiple patients. The data can be collected by the one or more ambulatory medical devices, among other data acquisition sensors or devices associated with the patient 1001. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 1008 may additionally include one or more locally configured clients or remote clients securely connected over the communication network 1007 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 1008, including the server and the interconnected clients, may also execute a follow-up scheme by sending follow-up requests to the one or more ambulatory medical devices, or by sending a message or other communication to the patient 1001 (e.g., the patient), clinician or authorized third party as a compliance notification.

The communication network 1007 can provide wired or wireless interconnectivity. In an example, the communication network 1007 can be based on the Transmission Control Protocol/Internet Protocol (TCP/IP) network communication specification, although other types or combinations of networking implementations are possible. Similarly, other network topologies and arrangements are possible.

One or more of the external device 1006 or the remote device 1008 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 1006 or the remote device 1008 can include a respective display unit for displaying the physiologic or functional signals, or alerts, alarms, emergency calls, or other forms of warnings to signal the detection of arrhythmias. In some examples, the external system 1005 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 1005 can be implemented using hardware, software, firmware, or combinations thereof. Portions of the one or more ambulatory medical devices or the external system 1005 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 1010 can be configured to send information to or receive information from one or more of the ambulatory medical devices or the external system 1005 using the communication link 1011. In an example, the one or more ambulatory medical devices, the external device 1006, or the remote device 1008 can be configured to control one or more parameters of the therapy device 1010. The external system 1005 can allow for programming the one or more ambulatory medical devices and can receives information about one or more signals acquired by the one or more ambulatory medical devices, such as can be received via a communication link 1011. The external system 1005 can include a local external implantable medical device programmer. The external system 1005 can include a remote patient management system that can monitor patient status or adjust one or more therapies such as from a remote location.

Ambulatory medical devices can additionally include or be configured to receive mechanical acceleration information from one or more accelerometer sensors to determine and monitor patient acceleration information, such as cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection.

In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.).

A timing metric between first and second cardiac features can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. The change in modes can enable higher resolution sampling or an increase in the sampling frequency or number or types of sensors used to sense physiologic information leading up to and including the potential event. For example, heart sounds and patient activity are often detected using non-overlapping time periods of the same, single- or multi-axis accelerometer, at different sampling frequencies and power costs. In one example, the transition to a high-power mode can include using the accelerometer to detect heart sounds throughout the high-power mode, or at a larger percentage of the high-power mode than during a corresponding low-power mode, etc. Additionally, waveforms for medical events are often recorded, stored in long-term memory, and frequently transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device. Accordingly, for numerous reasons, it is advantageous to accurately detect and determine physiologic events, including reducing false positive device detections, to properly manage and utilize medical device resources.

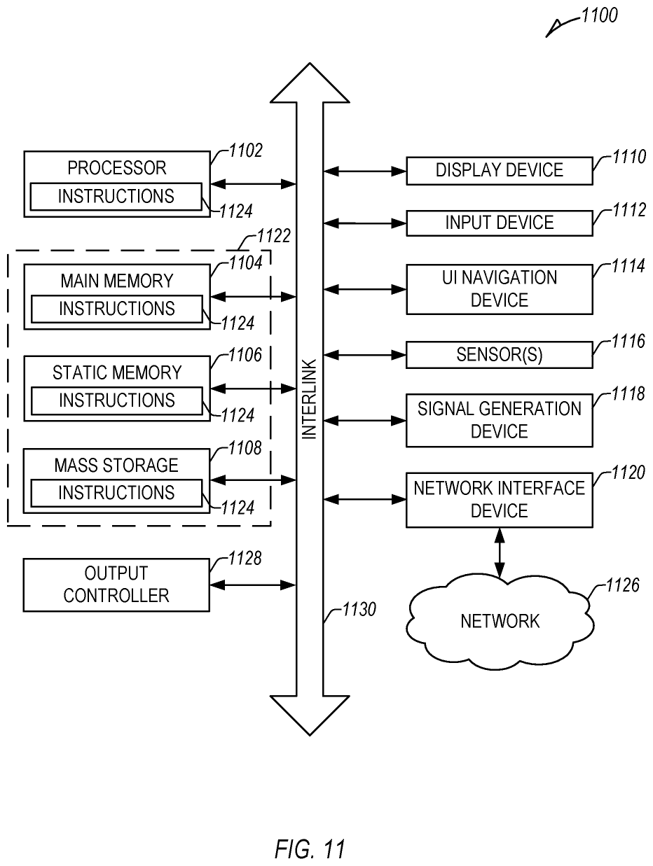

FIG. 11 illustrates a block diagram of an example machine 1100 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1100. Circuitry (e.g., processing circuitry, an assessment circuit, etc.) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1100 (e.g., computer system) may include a hardware processor 1102 (e.g., a central processing unit (CPU), a graphics processing unit (GPU), a hardware processor core, or any combination thereof), a main memory 1104, a static memory 1106 (e.g., memory or storage for firmware, microcode, a basic-input-output (BIOS), unified extensible firmware interface (UEFI), etc.), and mass storage 1108 (e.g., hard drive, tape drive, flash storage, or other block devices) some or all of which may communicate with each other via an interlink 1130 (e.g., bus). The machine 1100 may further include a display unit 1110, an input device 1112 (e.g., a keyboard), and a user interface (UI) navigation device 1114 (e.g., a mouse). In an example, the display unit 1110, input device 1112, and UI navigation device 1114 may be a touch screen display. The machine 1100 may additionally include a signal generation device 1118 (e.g., a speaker), a network interface device 1120, and one or more sensors 1116, such as a global positioning system (GPS) sensor, compass, accelerometer, or one or more other sensors. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 1102, the main memory 1104, the static memory 1106, or the mass storage 1108 may be, or include, a machine-readable medium 1122 on which is stored one or more sets of data structures or instructions 1124 (e.g., software) embodying or utilized by any one or more of the techniques or functions described herein. The instructions 1124 may also reside, completely or at least partially, within any of registers of the hardware processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine-readable medium 1122. While the machine-readable medium 1122 is illustrated as a single medium, the term “machine-readable medium” may include a single medium or multiple media (e.g., a centralized or distributed database, and/or associated caches and servers) configured to store the one or more instructions 1124.

The term “machine-readable medium” may include any medium that is capable of storing, encoding, or carrying instructions for execution by the machine 1100 and that cause the machine 1100 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1100, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive physiologic information of a patient, the physiologic information including cardiac acceleration information and blood pressure information of the patient; and
an assessment circuit configured to:
determine a syncope metric for the patient as a function of cardiac acceleration information and the blood pressure information;
determine an indication of a worsening risk of syncope or a syncope event for the patient using the determined syncope metric; and
provide the determined indication of the worsening risk of syncope or the syncope event for the patient to a user or a process.

2. The system of claim 1, wherein the cardiac acceleration information includes heart sound information of the patient, including second heart sound (S2) information,
wherein the blood pressure information comprises an indication of systolic blood pressure.

3. The system of claim 2, wherein the S2 information comprises a daily S2 value,
wherein the blood pressure information comprises a daily systolic blood pressure value.

4. The system of claim 1, wherein the assessment circuit is configured to determine the syncope metric using an increase in the cardiac acceleration information and a decrease in the blood pressure information,
wherein the assessment circuit is configured to determine the indication of the worsening risk of syncope or the syncope event for the patient using a comparison of the determined syncope metric to a threshold.

5. The system of claim 4, wherein the assessment circuit is configured to determine a baseline syncope metric using physiologic information of the patient,
wherein the assessment circuit is configured to determine the indication of the worsening risk of syncope or the syncope event for the patient using a comparison of the determined syncope metric to the baseline syncope metric.

6. The system of claim 1, comprising:
an ambulatory medical device including an accelerometer configured to sense the cardiac acceleration information; and
an external medical device including a pressure sensor configured to sense a systolic blood pressure of the patient, wherein the blood pressure information comprises the sensed systolic blood pressure information,
wherein the signal receiver circuit is configured to receive the cardiac acceleration information from the ambulatory medical device and the systolic blood pressure information from the external medical device,
wherein the assessment circuit is configured to provide a control to at least one of the ambulatory medical device or the external medical device using the determined indication of the worsening risk of syncope or the syncope event for the patient.

7. The system of claim 1, wherein the assessment circuit is configured to transition a medical device of the medical device system from a first low-power mode to a second high-power mode using the determined risk of syncope or the Syncope event for the patient.

8. The system of claim 7, wherein the first low-power mode comprises an ambulatory monitoring mode,
wherein the second high-power mode comprises a higher-power monitoring mode including storing received cardiac acceleration information and the received blood pressure information in long-term storage,
wherein the medical device comprises an implantable medical device comprising the signal receiver circuit and the assessment circuit.

9. The system of claim 1, wherein the assessment circuit is configured to determine the indication of the worsening risk of syncope or the syncope event for the patient using an increase in the cardiac acceleration information of the patient greater than a first threshold and a decrease in the blood pressure information of the patient greater than a second threshold.

10. The system of claim 1, wherein the assessment circuit is configured to detect a perturbation event using the received physiologic information or receiving an indication of the perturbation event,
wherein the assessment circuit is configured to determine the syncope metric for the patient using received cardiac acceleration information occurring over an early window of the detected perturbation event,
wherein the early window follows the detected perturbation event after a first blanking period.

11. The system of claim 1, wherein to provide the determined indication of the worsening risk of syncope or the syncope event for the patient to the user or the process comprises to provide a trigger to update the received blood pressure information in response to the determined indication of the worsening risk of syncope or the syncope event.

12. The system of claim 1, wherein the signal receiver circuit is configured to receive the cardiac acceleration information of the patient, including heart sound information of the patient, including at least two of: first heart sound (S1) information, second heart sound (S2) information, third heart sound (S3) information, and fourth heart sound (S4) information,
wherein, in response to the determined indication of the worsening risk of syncope or the syncope event for the patient, the assessment circuit is configured to determine a predicted fainting metric for the patient using the at least two of the first S1 information, the S2 information, the S3 information, and the S4 information.

13. The system of claim 1, comprising:
a therapy circuit configured to alter or provide one of a drug therapy or a pacing therapy in response to the determined indication of the worsening risk of syncope or the syncope event.

14. A method, comprising:
receiving physiologic information of a patient using a signal receiver circuit, the physiologic information including cardiac acceleration information and blood pressure information of the patient; and
using an assessment circuit:
determining a syncope metric for the patient as a function of cardiac acceleration information and the blood pressure information;
determining an indication of a worsening risk of syncope or a syncope event for the patient using the determined syncope metric; and
providing the determined indication of the worsening risk of syncope or the syncope event for the patient to a user or a process.

15. The method of claim 14, wherein the cardiac acceleration information includes heart sound information of the patient, including second heart sound (S2) information,
wherein the blood pressure information comprises an indication of systolic blood pressure.

16. The method of claim 15, wherein the S2 information comprises a daily S2 value,
wherein the blood pressure information comprises a daily systolic blood pressure value.

17. The method of claim 14, wherein determining the syncope metric comprises using an increase in the cardiac acceleration information and a decrease in the blood pressure information,
wherein determining the indication of the worsening risk of syncope or the syncope event for the patient comprises using a comparison of the determined syncope metric to a threshold.

18. The method of claim 17, comprising:
determining, using the assessment circuit, a baseline syncope metric using physiologic information of the patient,
wherein determining the indication of the worsening risk of syncope or the syncope event for the patient comprises using a comparison of the determined syncope metric to the baseline syncope metric.

19. The method of claim 14, comprising:
sensing the cardiac acceleration information using an accelerometer of an ambulatory medical device;
sensing a systolic blood pressure of the patient using a pressure sensor of an external medical device, wherein the blood pressure information comprises the sensed systolic blood pressure information; and
providing, using the assessment circuit, a control to at least one of the ambulatory medical device or the external medical device using the determined indication of the worsening risk of syncope or the syncope event for the patient,
wherein receiving the cardiac acceleration information comprises from the ambulatory medical device and receiving the systolic blood pressure information comprises from the external medical device.

20. The method of claim 14, comprising:
transitioning a medical device, using the assessment circuit, from a first low-power mode to a second high-power mode using the determined risk of syncope or the syncope event for the patient,
wherein the first low-power mode comprises an ambulatory monitoring mode,
wherein the second high-power mode comprises a higher-power monitoring mode including storing received cardiac acceleration information and the received blood pressure information in long-term storage.
